(19) United States
(12) Patent Application Publication
(10) Pub. No.: US 2005/0129664 A1
(43) Pub. Date: Jun. 16, 2005

(54) Remedy for dysmnesia

(76) Inventors: Hideyuki Okano, Tokyo (JP); Takuya Simakazi, Tokyo (JP); Seigo Nagao, Kagawa (JP); Yoshihito Matsumoto, Kagawa (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/499,825

(22) PCT Filed: Oct. 15, 2002
(86) PCT No.: PCT/JP02/10647

(30) Foreign Application Priority Data
Jan. 9, 2002 (JP) 2002-002433

(51) Int. Cl. A61K 45/00; C12N 5/08

(52) U.S. Cl. 424/93.7; 435/368

(57) ABSTRACT

Use of a culture of neural stem cells originating from embryonic stem cells in production of a remedy for dysmnesia. the present invention enables treatment of dysmnesia associated, for example, with Alzheimer's disease.

### TECHNICAL FIELD

The present invention relates to a remedy for dysmnesia caused by brain disorder such as Alzheimer's disease.

### BACKGROUND ART

Alzheimer's disease (as used herein, so-called Alzheimer's disease (AD) and senile dementia of the Alzheimer type. (SDAT), which develops following AD, are collectively included) is a decline in intellectual functioning where dementia or dysmnesia is a primary symptom. Among the theories offered in explanation of the cause, the beta-amyloid theory has come to be widely accepted. According to the beta-amyloid theory, a neurotoxin of β-amyloid protein, which is primarily found in senile plaques, induces synapse loss and neuron death.

Known drugs used to treat Alzheimer's disease include tacrine and donepezil, which are cholinesterase inhibitors. However, their effects are not necessarily satisfactory.

Accordingly, an object of the present invention is to provide a novel remedy for dysmnesia in, for example, Alzheimer's disease.

### DISCLOSURE OF THE INVENTION

Using dysmnesia model animals, the present inventors have studied transplant therapy with embryonic stem cells which have ability to differentiate into a variety of different cells, and have found that as reported previously, the embryonic stem cells were not suitable as transplant donor cells, because they exhibited tumorgenic propagation at the site of transplantation but that when neural stem cells derived from embryonic stem cells through culture are transplanted, significant improving effect on dysmnesia was obtained, leading to completion of the present invention.

Accordingly, the present invention provides use of a culture of neural stem cells originating from embryonic stem cells in producing a remedy for dysmnesia.

The present invention also provides a method for treating dysmnesia, characterized by administering an effective amount of a culture of neural stem cells originating from embryonic stem cells.

### BRIEF DESCRIPTION OF THE DRAWINGS

FIG. 1 shows the relation between days of culturing embryoid bodies and formation of neurospheres.

FIG. 2 shows the effect of added noggin protein.

FIG. 3 is a graph showing the results of a Morris's water maze test (WMT), which was performed to measure deterioration in memory and learning ability induced by ibotenic acid as well as recovery after transplant of cells.

FIG. 4 shows that administration of ibotenic acid to the mouse medial septal nucleus results in disappearance of ChAT-positive cells (cholinergic neurons). (Note that they were present in healthy mice, but absent in the ibotenic acid administration group.)

FIG. 5 shows that neural stem cells originating from ES cells and transplanted into the hippocampus have undergone cell division. (Compare between the groups GFP, BrDU, and (GFP+BrDU))

FIG. 6 shows that neural stem cells originating from ES cells and transplanted into the hippocampus have been differentiated into neurons. (Compare between the groups GFP, Hu, and (GFP+BrDU)).

FIG. 7 shows that neural stem cells originating from ES cells and transplanted into the hippocampus have been differentiated into cholinergic neurons. (The right image is an enlargement of the image on the left.)

FIG. 8 shows that neural stem cells originating from ES cells and transplanted into the hippocampus have sparingly been differentiated into GABAergic neurons. (Compare between the groups GFP, GAD, and (GFP+GAD)).

FIG. 9 shows that neural stem cells originating from ES cells and transplanted into the hippocampus have sparingly been differentiated into astrocytes. (Compare between the groups GFP, GFAP, and (GFP+GFAP)).

FIG. 10 shows that neural stem cells originating from ES cells and transplanted into the hippocampus can be differentiated into neurons and form synapses. (Compare between the groups GFP, synaptophysin, and (GFP+synaptophysin)).

FIG. 11 shows the results of immunohistochemical staining of the site of transplant six months after transplantation. The neural stem cells originating from ES cells and transplanted into the hippocampus have been differentiated into Hu-positive neurons (Compare between the groups ChAT and (GFP+ChAT)) and also into ChAT-positive cholinergic neurons (Compare between the groups GFP, ChAT and (GFP+ChAT)), proving their survival even after six months had elapsed from the transplantation.

FIG. 12 shows formation of a tumor which occurred after undifferentiated ES cells were transplanted into the hippocampus. (A) on the left shows the results of horizontal view of a brain with tumor, and (B) on the right shows an image of a slice.

### BEST MODE FOR CARRYING OUT THE INVENTION

The neural stem cells to be used in the present invention is derivable from embryonic stem cells. In an exemplary method for obtaining neural stem cells from embryonic stem cells (ES cells), embryonic stem cells are subjected to suspension culture in the presence or absence of a noggin protein, to thereby form embryoid bodies (EBs), which are then suspension-cultured in the presence of a fibloblast growth factor and a sonic hedgehog protein. Use of the thus-cultured neural stem cells is particularly preferred from the viewpoint of attainable remedial effect on dysmnesia.

ES cells useful in the present invention may be those which have already been established as cultured cells. For example, ES cell lines from mice, hamsters, pigs, and humans may be employed. Specific examples include 129/O1a-mouse-derived ES cells, such as EB3 and E14tg2. Preferably, the ES cells are subcultured in a serum-containing GMEM medium or a similar medium.

In formation of embryoid bodies from ES cells, suspension culture of ES cells in a medium to which a noggin protein has been added is effective for promoting differentiation-inducing efficiency from ES cells to neural stem cells. The noggin protein may be a Xenopus noggin protein. Alternatively, full-length cDNA of Xenopus noggin is transferred to COS7 cells, followed by culturing to cause transient expression of the noggin protein, and the resultant supernatant may be used as is. Preferably, the concentration of the noggin protein in a medium is 1 to 50% (v/v) or thereabouts in terms of the volume of culture supernatant. Suspension culture of ES cells is performed by use of serum-containing α-MEM medium for 4 to 8 days at a concentration of approximately 1×10⁵ ES cells/mL. Examples of useful sera include bovine serum and pig serum. The serum concentration is 5 to 15%, preferably 8 to 12%. Preferably, 2-mercaptoethanol is added to the α-MEM medium in such an amount that achieves a concentration of 0.01 to 0.5 mM, particularly 0.05 to 0.2 mM. The culturing is preferably performed in 5, % CO₂, at 35-40° C.

It is very preferred that the noggin protein be added during formation of embryoid bodies; i.e., during the period from day 1 to day 6 of culturing.

In order to amplify neural stem cells which have been obtained from ES cells via the above-prepared embryoid bodies, suspension culturing is performed by use of a neural stem cell amplification medium containing not only a fibroblast growth factor but also a sonic hedgehog protein.

Examples of preferred fibroblast growth factors (FGFs) are FGF-2 and FGF-8. The FGF content of the medium is preferably 5 to 50 ng/mL, more preferably 10 to 40 ng/mL. Examples of preferred sonic hedgehog proteins include mouse sonic hedgehog protein. The sonic hedgehog protein content of the medium is 1 to 20 nM, preferably 1 to 10 nM.

The medium is preferably a DMEM medium containing, in addition to the aforementioned components, glucose, glutamine, insulin, transferrin, progesterone, putrecine, selenium chloride, heparin, etc. Use of a DMEM:F12 medium is particularly preferred. Culturing is preferably performed in 5% CO₂, at 35-40° C., for a period of 7 to 9 days.

Through the above-described suspension culture, a single-cell-derived, aggregated mass of cells, called neurospheres, is formed. The thus-obtained neurospheres have originated solely from neural stem cells, and thus the above-mentioned culture method is proven to attain very high efficiency of differentiation into neural stem cells.

In the present invention, neural stem cells are preferably employed in the form of neurospheres prepared as described above.

The neural stem cell culture may contain, in addition to a variety of buffers, neurotrophic factors such as BDNF, CNTF, NGF, NT-3, and NT-4.

The neural stem cell culture exerts an effect of significantly improving dysmnesia caused by loss of cholinergic neurons, which often occurs in relation to Alzheimer's disease or brain atrophy developed after injury of the head, or after surgery of intra-brain lesions such as strokes and brain tumors. The manner of administration is preferably transplant into the site of an intra-brain lesion; for example, in the case of Alzheimer's disease, into the site of senile plaques. Transplant is preferably carried out after the lesion site has been identified by any suitable means such as MRI or CT scanning. The amount of neural stem cells to be transplanted differs depending on the condition of the patient and the size of the lesion site, but is preferably 1×10⁶ to 1×10⁸ cells per transplant for an adult.

In the treatment of dysmnesia by use of the remedy of the present invention for dysmnesia, other dysmnesia remedies, such as tacrine and donepezil, may be used in combination.

### EXAMPLES

The present invention will next be described in more detail by way of examples, which should not be construed as limiting the invention thereto.

### Production Example

A. Materials and Methods

(1) Subculture of Mouse ES Cells and Formation of Embryoid Bodies

E14tg2a ES cells derived from 129/O1a mice and EB3 ES cells (which allow selection of undifferentiated ES cells through insertion of blasticidin-resistant gene to the Oct3/4 locus of E14tg2a) were subcultured by a routine method in a GMEM medium (Glasgow minimum essential medium) containing 10% fetal calf serum, nonessential amino acids, 1 mM sodium pyruvate, 0.1 mM 2-mercaptoethanol, and 1,000 U/mL leukemia inhibitory factor (LIF). The culture conditions were 5% CO₂ at 37° C. (hereafter, when “culture” is referred to, these conditions apply).

Formation of embryoid bodies (EBs) from the ES cells was carried out as follows. Firstly, ES cells were washed with PBS. Subsequently, the washed cells were treated with 0.25% trypsin—1 mM EDTA, and then the treatment reaction was stopped. The cells were dissociated by pipetting, and seeded (1×10⁵ cells/mL) in a culture dish for bacteria filled with α-MEM medium containing 10% fetal calf serum and 0.1 mM 2-mercaptoethanol. In the presence or absence of noggin protein, suspension culture was performed for 4 to 8 days, whereby EBs were formed. The noggin protein employed was a culture supernatant of COS7 cells to which full-length cDNA of Xenopus noggin had been introduced for transient expression.

(2) Isolation of Neural Stem Cells by Selective Culture of EBs

The EBs formed as described above, together with the culture broth, were transferred to a centrifuge tube. The tube was left to stand for 10 minutes, to thereby allow the EBs to sediment at the bottom. The supernatant was removed, and the EBs were re-suspended in PBS. The test tube was left to stand for 10 minutes again. The supernatant was removed, and the EBs were re-suspended in a solution containing 0.25% trypsin and 1 mM EDTA PBS, followed by incubation at 37° C. for five minutes. Protein degradation reaction was stopped by use of α-MEM medium containing 10% fetal calf serum. The cells were dissociated by pipetting. The dissociated cells were centrifugally washed with α-MEM medium twice, and seeded at a concentration of 5×10⁴ cells/mL in either of the following mediums designed for neural cell amplification: a 1:1 medium of DMEM (Dulbecco's modified Eagle's medium) and F12, where the DMEM had been supplemented with glucose (0.6%), glutamine (2 mM), insulin (25 μg/mL), transferrin (100 μg/mL), progesterone (20 nM), putrecine (60 μM), selenium chloride (30 nM), FGF-2 (20 ng/mL), and heparin (2 μg/mL); or the same medium but further containing a mouse sonic hedgehog protein (mouse sonic hedgehog 1; 5 nM), followed by suspension culture for 7 to 9 days, whereby neurospheres (cell clusters derived from a single cell) were formed. The neurospheres were centrifugally washed with a differentiation medium having the above-described formula, except that neither FGF-2 nor heparin were contained, and the washed cells—in the “as washed” state or after dissociated through pipetting—were seeded in a culture petri dish coated with poly-L-ornithine and filled with a differentiation medium, whereby differentiation is allowed to proceed in the presence or absence of a sonic hedgehog protein (5 nM) for 5 to 7 days. Separately, the above-obtained neurospheres were again dissociated into single cells, subcultured in a medium designed for amplification of neural stem cells for 7 days, to thereby form secondary neurospheres. The thus-obtained secondary neurospheres are also caused to differentiate as described above.

B. Test Results

(1) Isolation and Purification of Neural Stem Cells Through Selective Culture of EBs

In connection with the early stage differentiation of ES cells where formation of EBs occurs, studies were performed to identify the stage in culturing where neural stem cells emerge. Briefly, EBs which had been cultured for 4 to 8 days were dissociated into single cells, followed by culturing for 7 days in a culture medium for amplifying neural stem cells, whereby neurospheres were formed. The thus-formed neurospheres were transferred to a differentiation medium for differentiation, and their differentiation capacity was determined. In addition, neurospheres were subcultured for checking their self-renewal capacity.

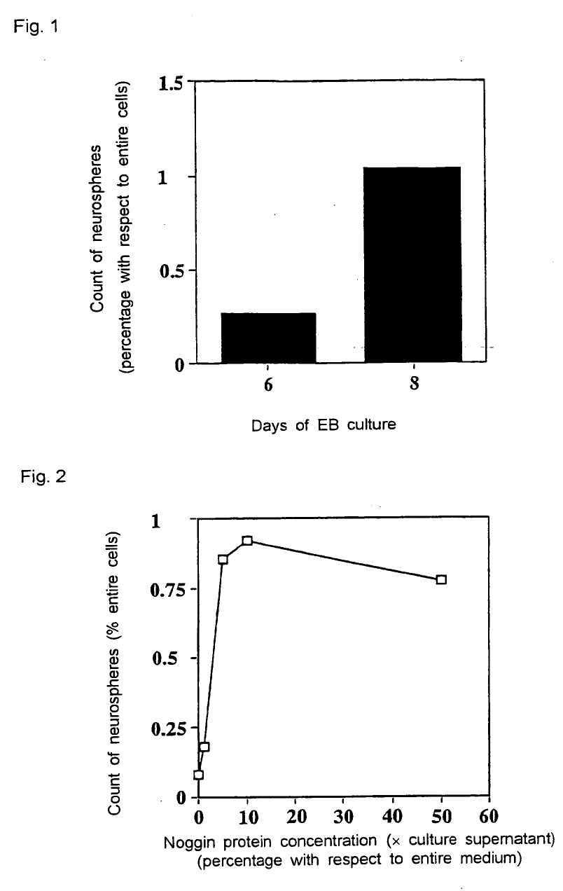

FIG. 1 shows the results of selective culture of neural stem cells (the neurosphere method), wherein 6 or 8 days after start of EB formation through suspension culture, the formed EBs were dissociated into single cells and subjected to the neurosphere method. The number of the neural stem cells that emerged among the EBs was taken as that of the obtained neurospheres. Neural stem cells (capable of forming neurospheres) which were to be identified by the present method were virtually not detected until day 4 of culture. On day 6 of culture, neural stem cells accounted 0.25% of all the cells, and on day 8, neural stem cells accounted 1.1%, thus gradual increase in cell count was acknowledged.

(2) Improvement of Efficiency in Inducing Neural Stem Cell Differentiation by Use of Noggin Protein

In an attempt to improve efficiency in inducing neural stem cell differentiation, during EB formation (6 days), noggin protein was added. The noggin protein employed was in the form of solution prepared by use of supernatant of the culture in which full-length cDNA of Xenopus had been integrated into a pEF-BOS expression vector and then transferred to COS7 cells for transient expression. The control employed was a supernatant of culture of COS7 cells to which only the expression vector had been incorporated. As shown in FIG. 2, the number of neural stem cells forming neurospheres which are caused to differentiate among EBs increases in accordance with the volume of the noggin culture supernatant, reaching a peak at 1/10 in volume.

### Example 1

Ibotenic acid (10 μg) was surgically administered to male mice (9 weeks old) at the medial septal nucleus thereof so as to disrupt cholinergic neurons, to thereby prepare dysmnesia model mice. To the hippocampus of each of the dysmnesia mice, neural stem cells obtained through inducing differentiation of ES cells to which GFP (green fluorescence protein) gene had been introduced were transplanted, and remedial effect on dysmnesia was studied. Specifically, the male mice (9 weeks old) were divided into the following four groups, and dysmnesia model mice were prepared through administration of ibotenic acid to the mice.

(1) Control Group

Administration of PBS (1 μL) to the medial septal nucleus+administration of PBS (1 μL) to the hippocampus

(2) Non-Treatment Group

Administration of ibotenic acid (1 μg) to the medial septal nucleus+administration of PBS (1 μL) to the hippocampus

(3) ES-Cell-Transplant Group

Administration of ibotenic acid (1 μg) to the medial septal nucleus+transplantation of ES cells to the hippocampus

(4) Group of Mice to Which Neural Stem Cells Obtained Through Differentiation of ES Cells Were Transplanted

Administration of ibotenic acid (1 μg) to the medial septal nucleus+transplantation of neural stem cells (1 μL) to the hippocampus

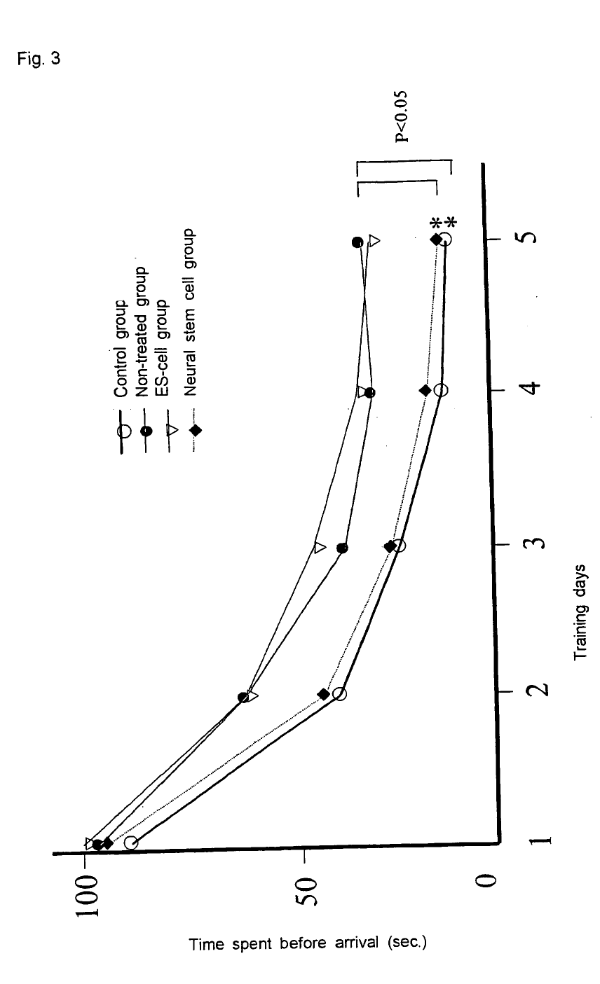

For each of the above groups, the effect of alleviating dysmnesia was studied through the Morris's water maze test (WMT). (Morris's water maze test: In a circular water bath (diameter: 1.8 m), a platform (diameter 10 cm) is submerged, and a mouse is released at a predetermined position on the water surface of the bath, to thereby measure the time required for the mouse to arrive at the platform. The measurement is repeated, to thereby determine the memory-based learning ability of the mouse.) As shown in FIG. 3, the mice to which neurospheres of neural stem cells were transplanted were found to have recovered from the dysmnesia to the same extent as had the mice of the control group. In contrast, as had been reported earlier, the mice to which ES cells were transplanted were observed to have proliferated tumor cells, revealing that the ES cells are not suitable as donor cells for transplantation.

The transplant portions were stained immunohistochemically to investigate the state of differentiation of neurons.

Details of the immunostaining are as follows: anti-GFP antibody was employed to identify transplanted cells and progeny cells thereof; anti-BrdU antibody was employed to investigate whether or not transplanted cells had been divided (specifically, BrdU (120 mg/kg) was administered to a host mouse that had undergone transplant, and cells that exhibited intake of BrdU were detected); anti-Hu antibody was employed to investigate whether or not the cells had been differentiated to neurons; anti-ChAT antibody was employed to investigate whether or not the cells were differentiated to cholinergic neurons; anti-GAD67 antibody was employed to investigate whether or not the cells had been differentiated to GABA neurons; anti-GFAP antibody was employed to investigate whether or not the cells had been differentiated to astroglial; and anti-synaptophysin antibody was employed to investigate whether or not neurons derived from transplanted cells had formed synapses.

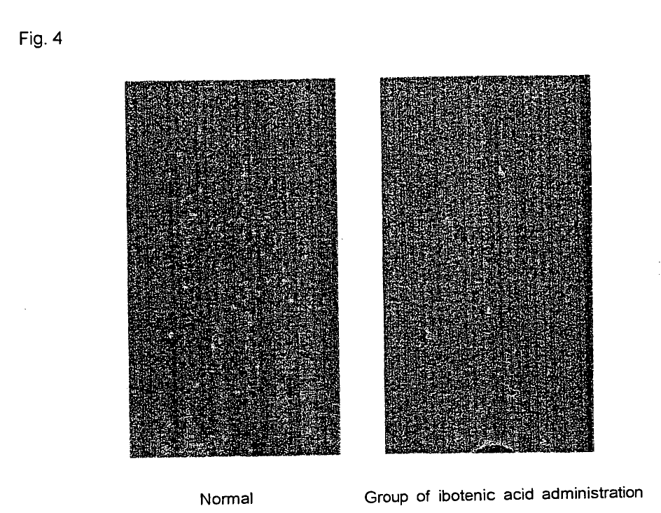

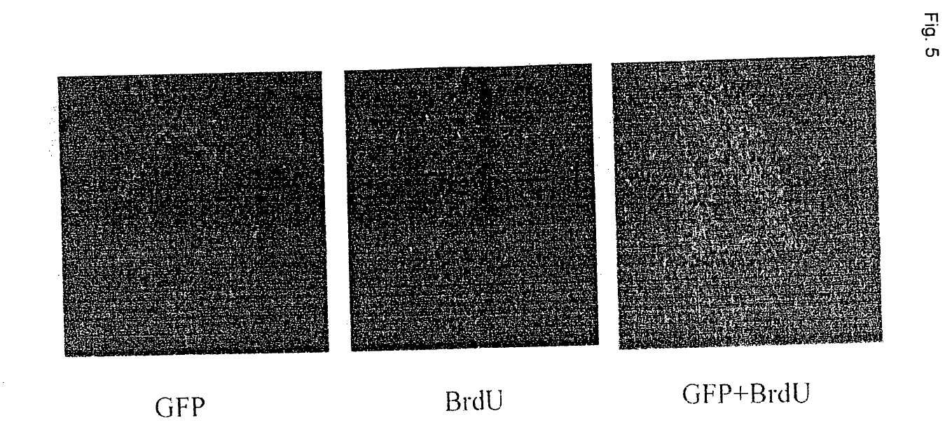

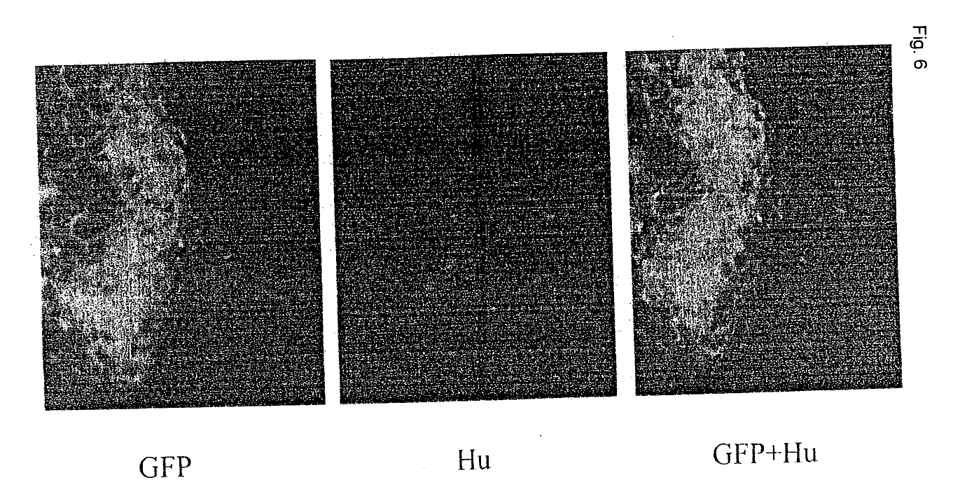

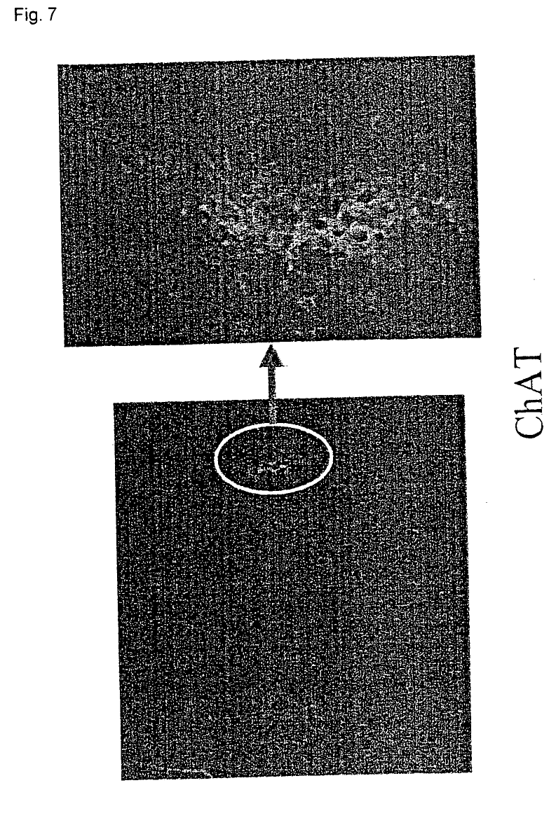

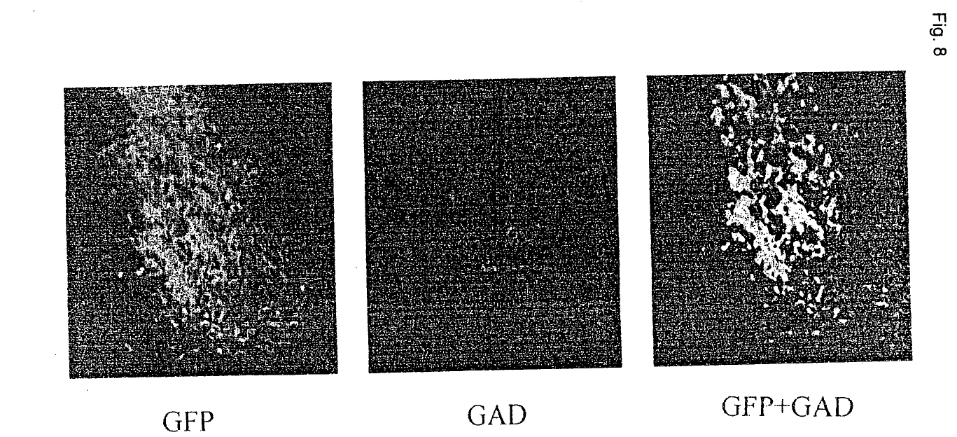

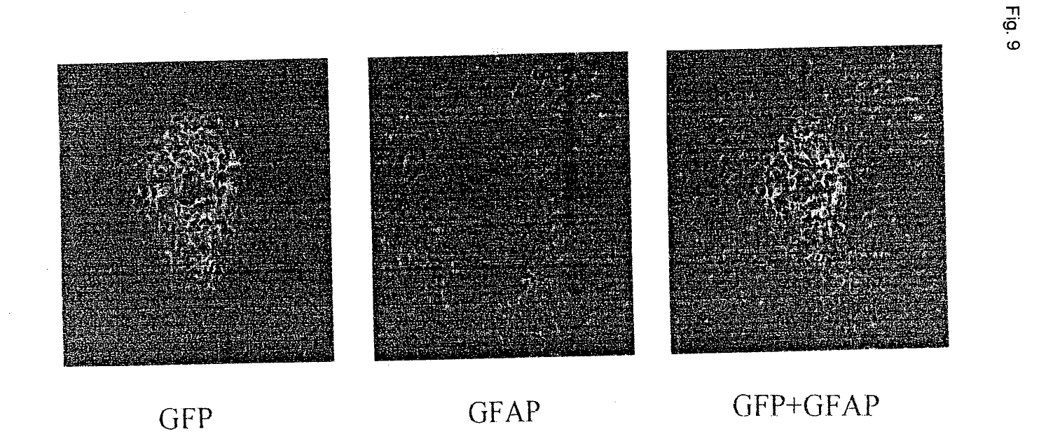

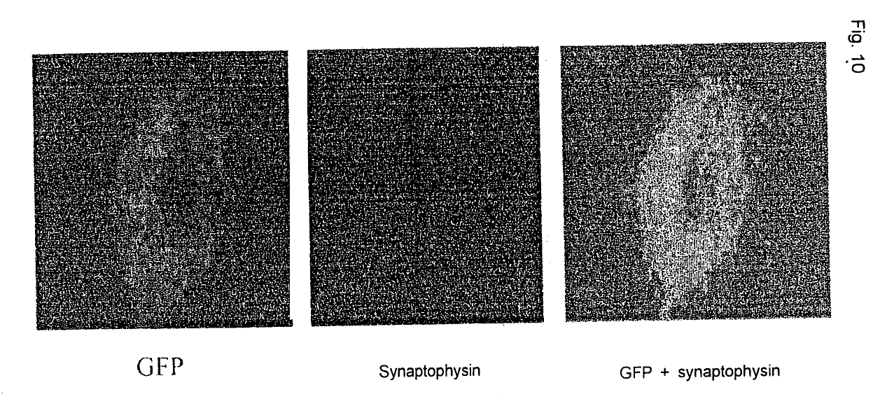

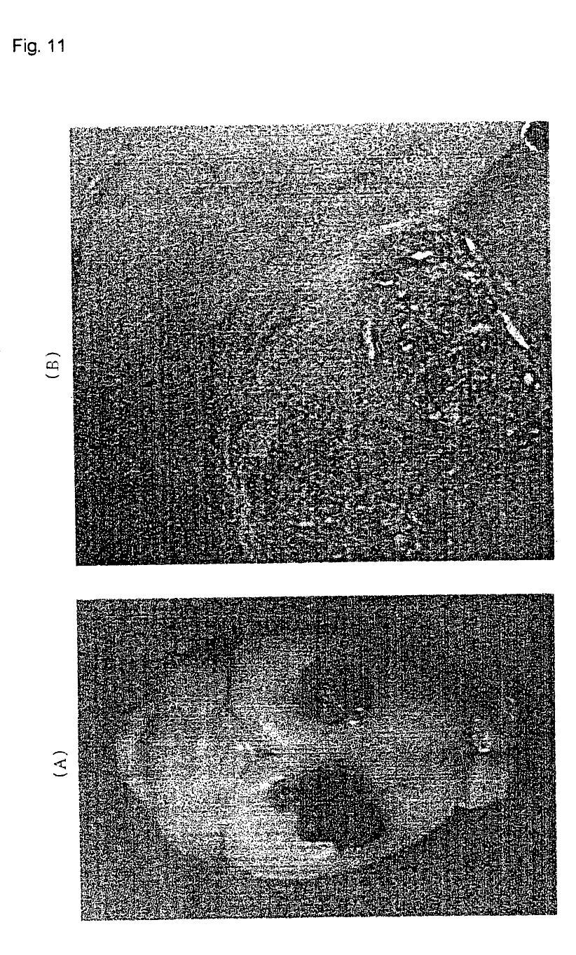

The results are shown in FIGS. 4 to 12. As is clear from FIG. 4, in the medial septal nucleus of the dysmnesia mice, to which ibotenic acid had been administered, virtually no ChAT-positive cells (cholinergic neurons) were observed. As is clear from FIG. 5, most of the transplanted cells (GFP-positive cells) were taking up BrdU, revealing that transplanted cells had been divided. FIG. 6 shows that the transplanted cells can be differentiated to Hu-positive neurons. FIG. 7 shows that the transplanted cells are differentiated to ChAT-positive cholinergic neurons. FIG. 8 shows that, in the transplanted cells, a slight amount of GAD67-positive GABA-active neurons was detected. FIG. 9 shows that most of the cells transplanted to the hippocampus are not differentiated to astrocytes. FIG. 10 shows that neurons derived from the transplanted cells have the ability to form synapses. FIG. 11 shows the results of immunohistochemical staining of the site of transplant six months after transplant. The cells transplanted to the hippocampus were found to have been differentiated to Hu-positive neurons and ChAT-positive cholinergic neurons and to have survived even after six months had elapsed from the transplant. FIG. 12 shows that, when ES cells are transplanted without undergoing in vitro differentiation, tumors may be formed.

Industrial Applicability

The present invention enables treatment of dysmnesia associated, for example, with Alzheimer's disease.

1. Use of a culture of neural stem cells originating from embryonic stem cells in production of a remedy for dysmnesia.

2. The use according to claim 1, wherein the culture of neural stem cells originating from embryonic stem cells is obtained by subjecting embryonic stem cells to suspension culture in the presence or absence of a noggin protein, to thereby form embryoid bodies, and subsequently subjecting the embryoid bodies to suspension culture in the presence of a fibroblast growth factor and a sonic hedgehog protein.

3. The use according to claim 1, wherein the culture of neural stem cells originating from embryonic stem cells is obtained by subjecting embryonic stem cells to suspension culture in the presence of a noggin protein in an amount of 1 to 50% (v/v), to thereby form embryoid bodies, and subsequently subjecting the embryoid bodies to suspension culture in the presence of a fibroblast growth factor in an amount of 5 to 50 ng/mL and a sonic hedgehog protein in an amount of 1 to 20 nM.

4. The use according to any one of claims 1 through 3, wherein the culture of neural stem cells comprises neurospheres.

5. The use according to any one of claims 1 through 4, wherein the dysmnesia is associated with Alzheimer's disease, brain atrophy developed after injury of the head, cerebral apoplexy, or a sequela of brain surgery.

6. A method for treating dysmnesia, characterized by administering to a subject in need thereof an effective amount of a culture of neural stem cells originating from embryonic stem cells.

7. The method according to claim 6, wherein the culture of neural stem cells originating from embryonic stem cells is obtained by subjecting embryonic stem cells to suspension culture in the presence or absence of a noggin protein, to thereby form embryoid bodies, and subsequently subjecting the embryoid bodies to suspension culture in the presence of a fibroblast growth factor and a sonic hedgehog protein.

8. The method according to claim 6, wherein the culture of neural stem cells originating from embryonic stem cells is obtained by subjecting embryonic stem cells to suspension culture in the presence of a noggin protein in an amount of 1 to 50% (v/v), to thereby form embryoid bodies, and subsequently subjecting the embryoid bodies to suspension culture in the presence of a fibroblast growth factor in an amount of 5 to 50 ng/mL and a sonic hedgehog protein in an amount of 1 to 20 nM.

9. The method according to claim 6 or 7, wherein the culture of neural stem cells comprises neurospheres.

10. The method according to claim 6 or 7, wherein the dysmnesia is associated with Alzheimer's disease, brain atrophy developed after injury of the head, cerebral apoplexy, or a sequela of brain surgery.

11. The method according to claim 6, wherein administration is performed by means of intrabrain transplant.

12. A remedy for dysmnesia, containing a culture of neural stem cells originating from embryonic stem cells.

13. The remedy for dysmnesia according to claim 12, wherein the culture of neural stem cells originating from embryonic stem cells is obtained by subjecting embryonic stem cells to suspension culture in the presence or absence of a noggin protein, to thereby form embryoid bodies, and subsequently subjecting the embryoid bodies to suspension culture in the presence of a fibroblast growth factor and a sonic hedgehog protein.

14. The remedy for dysmnesia according to claim 12, wherein the culture of neural stem cells originating from embryonic stem cells is obtained by subjecting embryonic stem cells to suspension culture in the presence of a noggin protein in an amount of 1 to 50% (v/v), to thereby form embryold bodies, and subsequently subjecting the embryoid bodies to suspension culture in the presence of a fibroblast growth factor in an amount of 5 to 50 ng/mL and a sonic hedgehog protein in an amount of 1 to 20 nM.

15. The remedy for dysmnesia according to any one of claims 12 through 14, wherein the culture of neural stem cells comprises neurospheres.

16. The remedy for dysmnesia according to any one of claims 12 to 15, wherein the dysmnesia is associated with Alzheimer's disease, brain atrophy developed after injury of the head, cerebral apoplexy, or a sequela of brain surgery.
